(19) United States
(12) Patent Application Publication
(10) Pub. No.: US 2004/0101553 A1
(43) Pub. Date: May 27, 2004

(54) Platinum aggregates and process for producing the same

(75) Inventors: Jin Lee, Belle Mead, NJ (US); Brian S. Miller, Mercerville, NJ (US); Fangjun Wu, Livingston, NJ (US); Lawrence T. Boni, Monmouth Junction, NJ (US)

Correspondence Address: JOHN W. RYAN, C/O DECHERT LLP, PRINCETON PIKE CORPORATION CENTER, P.O. BOX 5218, PRINCETON, NJ, 08543-5218, US

(73) Assignee: Transave, Inc., Monmouth Junction, NJ

(21) Appl. No.: 10/634,144

(22) Filed: Aug. 4, 2003

Related U.S. Application Data
(60) Provisional application No. 60/400,875, filed on Aug. 2, 2002.

(51) Int. Cl. A61K 33/24; A61K 9/127

(52) U.S. Cl. 424/450; 424/649

(57) ABSTRACT

A composition and process for making the composition, the composition comprising a liposome and active platinum compound, the liposome containing one or more lipids, with a high active platinum compound to lipid ratio.

This application claims the priority of U.S. Provisional Application 60/400,875, filed Aug. 2, 2002.

Liposomes and lipid complexes have been long recognized as drug delivery systems which can improve therapeutic and diagnostic effectiveness of many bioactive agents and contrast agents. Experiments with a number of different antibiotics and X-ray contrast agents have shown that better therapeutic activity or better contrast with a higher level of safety can be achieved by encapsulating bioactive agents and contrast agents with liposomes or lipid complexes. Research on liposomes and lipid complexes as encapsulating systems for bioactive agents has revealed that a successful development and commercialization of such products requires reproducible methods of large scale production of lipid vesicles with suitable characteristics. Consequently, workers have searched for methods which consistently produce liposomes or lipid complexes of the required size and concentration, size distribution and, importantly, entrapping capacity, with flexible lipid composition requirements. Such methods ought to provide liposomes or lipid complexes with consistent active substance to lipid ratio while respecting currently accepted good manufacturing practices for pharmaceutical products. As a result of the search, and due to the variability of liposome and lipid complex behavior with production parameters, many different manufacturing methods have been proposed so far.

Conventional liposome and lipid complex preparation methods include a number of steps in which the bilayer-forming components (typically phospholipids or mixtures of phospholipids with other lipids e.g., cholesterol) are dissolved in a volatile organic solvent or solvent mixture in a round bottom flask followed by evaporation of the solvent under conditions, such as temperature and pressure, which will prevent phase separation. Upon solvent removal a dry lipid mixture, usually in form of a film deposit on the walls of the reactor, is hydrated with an aqueous medium which may contain dissolved buffers, salts, conditioning agents and an active substance to be entrapped. Liposomes or lipid complexes form in the hydration step such that a proportion of the aqueous medium becomes encapsulated in the liposomes. The hydration can be performed with or without energizing the solution by means of stirring, sonication or microfluidization or with subsequent extrusion through one or more filters, such as polycarbonate filters. The free non-encapsulated active substance can be separated for recovery and the product is filtered, sterilized, optionally lyophilized, and packaged.

In general, more than any other step in this conventional process, hydration can influence the type of liposomes or lipid complexes formed (size, number of lipid layers, entrapped volume). Hydration and the entrapping process are typically most efficient when the film of dry lipids is kept thin. This means that greater the lipid quantity, the greater the surface for deposition of the lipids that is required. Even though glass beads and other inert insoluble particles can be used to increase the surface area available for film deposition, the thin film method remains largely a laboratory method.

Other methods of making liposomes or lipid complexes involving injection of organic solutions of lipids into an aqueous medium with continuous removal of solvent, use of spray drying, lyophilization, microemulsification and microfluidization, and the like have been proposed in a number of publications or patents. Such patents include, for example, U.S. Pat. No. 4,529,561 and U.S. Pat. No. 4,572,425.

Cisplatin—cis-diamine-dichloroplatinum (II)—is one of the more effective anti-tumor agents used in the systemic treatment of cancers. This chemotherapeutic drug is highly effective in the treatment of tumor models in laboratory animals and in human tumors, such as endometrial, bladder, ovarian and testicular neoplasms, as well as squamous cell carcinoma of the head and neck (Sur, et al., 1983 Oncology 40(5): 372-376; Steerenberg, et al., 1988 Cancer Chemother Pharmacol. 21(4): 299-307). Cisplatin is also used extensively in the treatment of lung carcinoma, both SCLC and NSCLC (Schiller et al., 2001 Oncology 61(Suppl 1): 3-13). Other active platinum compounds (defined below) are useful in cancer treatment.

Like other cancer chemotherapeutic agents, active platinum compounds such as cisplatin are typically highly toxic. The main disadvantages of cisplatin are its extreme nephrotoxicity, which is the main dose-limiting factor, its rapid excretion via the kidneys, with a circulation half life of only a few minutes, and its strong affinity to plasma proteins (Freise, et al., 1982 Arch Int Pharmacodyn Ther. 258(2): 180-192).

Attempts to minimize the toxicity of active platinum compounds have included combination chemotherapy, synthesis of analogues (Prestayko et al., 1979 Cancer Treat Rev. 6(1): 17-39; Weiss, et al., 1993 Drugs. 46(3): 360-377), immunotherapy and entrapment in liposomes (Sur, et al., 1983; Weiss, et al., 1993). Antineoplastic agents, including cisplatin, entrapped in liposomes have a reduced toxicity, relative to the agent in free form, while retaining antitumor activity (Steerenberg, et al., 1987; Weiss, et al., 1993).

Cisplatin, however, is difficult to efficiently entrap in liposomes or lipid complexes because of the bioactive agent's low aqueous solubility, approximately 1.0 mg/ml at room temperature, and low lipophilicity, both of which properties contribute to a low bioactive agent/lipid ratio.

Liposomes and lipid complexes containing cisplatin suffer from another problem—stability of the composition. In particular, maintenance of bioactive agent potency and retention of the bioactive agent in the liposome during storage are recognized problems (Freise, et al., 1982; Gondal, et al., 1993; Potkul, et al., 1991 Am J Obstet Gynecol. 164(2): 652-658; Steerenberg, et al., 1988; Weiss, et al., 1993) and a limited shelf life of liposomes containing cisplatin, on the order of several weeks at 4° C., has been reported (Gondal, et al., 1993 Eur J Cancer. 29A(11): 1536-1542; Potkul, et al., 1991).

### SUMMARY OF THE INVENTION

Described is a new form of lipid-entrapped platinum and a method for producing the same. More particularly, described is a new form of lipid-complexed active platinum with a high active platinum compound to lipid ratio. The process described is a new process for forming this new form of a active platinum compound aggregate.

Provided, among other things, is a composition comprising a liposome or lipid complex and an active platinum compound, the liposome containing one or more lipids, wherein the active platinum compound to lipid ratio is from 1:50 to 1:2 by weight, or from 1:50 to 1:5 by weight, or from 1:50 to 1:10 by weight. The active platinum compound to lipid ratio can be, for example, from 1:25 to 1:15 by weight. The one or more lipids can comprise, for example, 50-100 mol % DPPC and 0-50 mol % cholesterol. The one or more lipids can comprise, for example, 50-65 mol % DPPC and 35-50 mol % cholesterol.

Also provided is a process for making a platinum aggregate comprising the steps of: (a) combining an active platinum compound and a hydrophobic matrix carrying system; (b) establishing the mixture at a first temperature; and (c) thereafter establishing the mixture at a second temperature, which second temperature is cooler than the first temperature; wherein the steps (b) and (c) are effective to increase the encapsulation of active platinum compound. Step (b) is typically effected with heating, while step (c) is typically effected with cooling. In alternative embodiments, the cycles are counted beginning with the cooler step, transitioning to the warmer step, and cycling the two steps. The process can comprise sequentially repeating the steps (b) and (c) for a total of two or three or more cycles. The active platinum compound solution can be produced by dissolving active platinum compound in a saline solution to form a platinum solution. The hydrophobic matrix carrying system favorably comprises liposome or lipid complex-forming lipids. The process for making a platinum aggregate can further comprise, after all of steps (b) and steps (c) have been completed: (d) removing un-entrapped active platinum compound by filtering through a membrane having a molecular weight cut-off selected to retain desired liposomes or lipid complexes and adding a liposome or lipid complex compatible liquid to wash out un-entrapped active platinum compound.

Provided further are aggregates produced by the methods of the invention and pharmaceutical formulations of the compositions of the invention. The formulations comprise pharmaceutically acceptable carrier or diluent or are adapted for delivery to a patient by inhalation or injection.

### DESCRIPTION OF THE DRAWING

FIG. 1 shows stability of one liter batches of lipid-complexed cisplatin according to the invention.

### DESCRIPTION OF THE INVENTION

The present invention comprises a new form of lipid-complexed active platinum compound which allows for a very high bioactive agent to lipid ratio, such as previously unseen with the active platinum compound cisplatin. The bioactive agent to lipid ratio seen in the present invention is between 1:5 by weight and 1:50 by weight. More preferably the bioactive agent to lipid ratio seen is between 1:10 by weight and 1:30 by weight. Most preferably the bioactive agent to lipid ratio seen is between 1:15 by weight and 1:25 by weight.

The process for producing this active platinum compound formulation can comprise mixing active platinum compound with an appropriate hydrophobic matrix and subjecting the mixture to one or more cycles of establishing two separate temperatures. The process is believed to form of an active platinum compound aggregate.

In aqueous solution, cisplatin forms large crystalline aggregates with a crystal diameter of greater than a few microns. In the presence of a amphipathic matrix system, such as a lipid bilayer, small cisplatin aggregates form. For example, the aggregates may be formed in the hydrocarbon core region of a lipid bilayer. During the warming cycle of the process, it is believed that cisplatin is returned to solution at a greater rate in aqueous regions of the process mixture than in the bilayers. As a result of applying more than one cool/warm cycle, cisplatin accumulates further in the bilayers. Without limiting the invention to the proposed theory, experimentation indicates that the cisplatin aggregates cause the immediate surroundings of the interfacial bilayer region to be more hydrophobic and compact. This results in a high level of entrapment of active platinum compound as cooling and warming cycles are repeated.

The formulation has a markedly high entrapment percentage. The entrapment has been shown, in some cases, to reach almost 92%. This amount is far higher than the most efficient entrapment expected from a conventional aqueous entrapment which is approximately 2-10% entrapment. This efficiency of the present invention is demonstrated in example 3.

The process comprises combining the bioactive agent with a hydrophobic matrix carrying system and cycling the solution between a warmer and a cooler temperature. Preferably the cycling is performed more than one time. More preferably the step is performed two or more times, or three or more times. The cooler temperature portion of cycle can, for example, use a temperature from −25 degrees Celsius and 25 degrees Celsius. More preferably the step uses a temperature from −5 and 5 degrees Celsius or between 1 and 5 degrees Celsius. For manufacturing convenience, and to be sure the desired temperature is established, the cooler and warmer steps can be maintained for a period of time, such as approximately form 5 to 300 minutes or 30 to 60 minutes. The step of warming comprises warming the reaction vessel to from 4 and 70 degrees Celsius. More preferably the step of warming comprises heating the reaction vessel to from 45 and 55 degrees Celsius. The above temperature ranges are particularly preferred for use with lipid compositions comprising predominantly diphosphatidycholine (DPPC) and cholesterol.

Another way to consider the temperature cycling is in terms of the temperature differential between the warmer and the cooler steps of the cycle. This temperature differential can be, for example, 25 degrees Celsius or more, such as a differential from 25 to 70 degrees Celsius, preferably a differential from 40 to 55 degrees Celsius. The temperatures of the cooler and higher temperature steps are selected on the basis of increasing entrapment of active platinum compound. Without being limited to theory, it is believed that it is useful to select an upper temperature effective substantially increase the solubility of active platinum compound in the processed mixture. Preferably, the warm step temperature is 50 degrees Celsius or higher. The temperatures can also be selected to be below and above the transition temperature for a lipid in the lipid composition.

The temperatures appropriate for the method may, in some cases, vary with the lipid composition used in the method, as can be determined by ordinary experimentation.

The resultant active platinum complex has a high or very high drug to lipid ratio. The formulation can be adapted for use by inhalation or injection.

For the purposes of this disclosure the following terms of art are used:

“Solvent infusion” is a process that includes dissolving one or more lipids in a small, preferably minimal, amount of a process compatible solvent to form a lipid suspension or solution (preferably a solution) and then injecting the solution into an aqueous medium containing bioactive agents. Typically a process compatible solvent is one that can be washed away in a aqueous process such as dialysis. The composition that is cool/warm cycled is preferably formed by solvent infusion, with ethanol infusion being preferred. Alcohols are preferred as solvents. “Ethanol infusion,” a type of solvent infusion, is a process that includes dissolving one or more lipids in a small, preferably minimal, amount of ethanol to form a lipid solution and then injecting the solution into an aqueous medium containing bioactive agents. A “small” amount of solvent is an amount compatible with forming liposomes or lipid complexes in the infusion process.

A “hydrophobic matrix carrying system” is the lipid/solvent mixture produced by the solvent infusion process described above.

The lipids used in the present invention can be synthetic, semi-synthetic or naturally-occurring lipids, including phospholipids, tocopherols, sterols, fatty acids, glycolipids, negatively-charged lipids, cationic lipids. In terms of phosholipids, they can include such lipids as egg phosphatidylcholine (EPC), egg phosphatidylglycerol (EPG), egg phosphatidylinositol (EPI), egg phosphatidylserine (EPS), phosphatidylethanolamine (EPE), and phosphatidic acid (EPA); the soya counterparts, soy phosphatidylcholine (SPC); SPG, SPS, SPI, SPE, and SPA; the hydrogenated egg and soya counterparts (e.g., HEPC, HSPC), stearically modified phosphatidylethanolamines, cholesterol derivatives, carotinoids, other phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The chains on these fatty acids can be saturated or unsaturated, and the phospholipid may be made up of fatty acids of different chain lengths and different degrees of unsaturation. In particular, the compositions of the formulations can include DPPC, a major constituent of naturally-occurring lung surfactant. Other examples include dimyristoylphosphatidycholine (DMPC) and dimyristoylphosphatidylglycerol (DMPG) dipalmitoylphosphatidcholine (DPPC and dipalmitoylphosphatidylglycerol (DPPG) distearoylphosphatidylcholine (DSPC and distearoylphosphatidylglycerol (DSPG), dioleylphosphatidyl-ethanolamine (DOPE) and mixed phospholipids like palmitoylstearoylphosphatidyl-choline (PSPC) and palmitoylstearolphosphatidylglycerol (PSPG), triacylglycerol, diacylglycerol, seranide, sphingosine, sphingomyelin and single acylated phospholipids like mono-oleoyl-phosphatidylethanolarnine (MOPE).

A “bioactive agent” is a substance that can act on a cell, virus, tissue, organ or organism to create a change in the functioning of the cell, virus, tissue, organ or organism. In the present disclosure the bioactive agent envisaged is an active platinum, such as cisplatin.

An “active platinum” compound is a compound containing coordinated platinum and having antineoplastic activity. Additional active platinum compounds include, for example, carboplatin and DACH-platinum compounds such as oxaliplatin.

Experimental results strongly indicate that encapsulation was achieved predominantly by capturing cisplatin during formation of liposomal vesicles. The results further indicate the physical state of cisplatin to be solid (aggregates) or lipid bound since the concentration of cisplatin is much higher than the solubility limit. Results further indicate that process does not require freezing the compositions, but that cooling to temperature higher than freezing can produce superior results. Results further indicated that an entrapment efficiency achieved by 3-cycles was similar to that achieved by 6-cycles of cooling and warming cycles, which indicated that 3 cycles of temperature treatment was sufficient to achieve highly preferred levels of entrapment.

Results further indicate that the process can be scaled-up while increasing process efficiency in entrapping cisplatin. Thus, the invention further provides processes that are conducted to provide an amount adapted for total administration (in appropriate smaller volume increments) of 200 or more mLs, 400 or more mLs, or 800 or more mLs. All else being the same, it is believed that the larger production volumes generally achieve increased efficiency over smaller scale processes. While such volume is that appropriate for administration, it will be recognized that the volume can be reduced for storage.

Results further indicate that the lipid-complexed cisplatin made by the method of the invention can retain entrapped cisplatin with minimal leakage for over one year. This is a further demonstration of the uniqueness in the formulation, indicating that the cisplatin is bound within the liposome structure and not free to readily leak out.

### EXAMPLES

### Example 1

70 mg DPPC and 28 mg cholesterol was dissolved in 1 ml ethanol and added to 10 ml of 4 mg/ml cisplatin in 0.9% saline solution.

(i) An aliquot (50%) of the sample was treated by 3 cycles of cooling to 4° C. and warming to 50° C. The aliquot, in a test tube, was cooled by refrigeration, and heated in a water bath. The resulting unentrapped cisplatin (free cisplatin) was washed by dialysis.

(ii) The remainder of the sample was not treated by temperature cycles and directly washed by dialysis.

TABLE 1
Percentage entrapment of cisplatin with
and without cooling and warming cycles

| | Final Concentration of | |
|---|---|---|
| | cisplatin, μg/ml | % Entrapment |
| Lipid-complexed cisplatin | 56 | 1.4 |
| without cooling and warming | | |
| cycles | | |
| lipid-complexed cisplatin after | 360 | 9.0 |
| cooling and warming cycles | | |

### Example 2

The rigidity of membrane bilayer in lipid-complexed cisplatin prepared with cool/warm cycling (“HLL” cisplatin or “high load liposomal” cisplatin) as described in Example 1 was measured by fluorescence anisotropy of diphenylhexatriene (membrane probe) inserted in the hydrophobic core region of the bilayer. [Ref. Jahnig, F., 1979 Proc. Natl. Acad. Sci. USA 76(12): 6361.] The hydration of the bilayers was gauged by the deuterium isotope exchange effect on fluorescence intensity of TMA-DPH (trimethylamine-diphenylhexatriene). [Ref. Ho, C., Slater, S. J., and Stubbs, C. D., 1995 Biochemistry 34: 6188.]

TABLE 2
Degree of hydration and rigidity of liposomes, lipid-complexed
cisplatin without cool/warm cycling and HLL cisplatin.

| | Placebo | Lipid-complexed | |
|---|---|---|---|
| | (Liposomes | cisplatin | |
| | without | without cooling & | |
| | cisplatin) | warming cycles | HLL cisplatin |
| Degree of rigidity of | 0.29 | 0.29 | 0.36 |
| bilayers | | | |
| Degree of hydration in | 1.13 | 1.15 | 1.02 |
| bilayers | | | |

### Example 3

1.0 g DPPC and 0.4 g cholesterol were dissolved in 6 ml of ethanol. 60 mg of cisplatin was dissolved in 10 ml of 0.9% saline solution at 65° C. 1 ml of the resultant lipid mixture solution was added to 10 ml of the resultant cisplatin solution. The lipid/cisplatin suspension was cooled to approximately 4° C. and held at that temperature for 20 min. and warmed to 50° C. and held at that temperature for 20 min. Ethanol was removed by bubbling N₂ gas into the suspension during the warming period. The cooling and warming steps were repeated 5 further times.

TABLE 3
Entrapment of cisplatin

| | Concentration of Total | % Cisplatin | Drug:Lipid |
|---|---|---|---|
| | Cisplatin (mg/ml) | entrapped | (by weight) |
| HLL Cisplatin | 5.8 | 91.6 | 1:26 |

### Example 4

A liposomal formulation was prepared using phosphatidylcholine (PC) and cholesterol (in a 57:43 mol ratio). 0.55 mmoles of PC and 0.41 mmoles of cholesterol were dissolved in 2 ml ethanol and added to 20 ml of 4 mg/ml cisplatin solution. An aliquot (50%) of each sample was treated by 3 cycles of cooling and warming and then washed by dialysis. Another part of each sample was directly washed by dialysis. Entrapment was estimated from the ratio of final concentration and initial concentration.

TABLE 4
Entrapment and drug to lipid ratios for cisplatin with
various phosphatidylcholines

| | No Cooling and Warming | | | Cooling and Warming | | |
|---|---|---|---|---|---|---|
| | | | Drug: | | | Drug: |
| | Final | % | Lipid | Final | % | Lipid |
| | [Cisplatin] | Entrap- | (by | [Cisplatin] | Entrap- | (by |
| PC | (mg/ml) | ment | weight) | (mg/ml) | ment | weight) |
| DOPC | 0.16 | 4.0 | 1:142 | 0.21 | 5.3 | 1:108 |
| EggPC | 0.09 | 2.3 | 1:247 | 0.12 | 3.0 | 1:185 |
| DMPC | 0.15 | 3.8 | 1:123 | 0.24 | 6.0 | 1:77 |
| DPPC | 0.17 | 4.3 | 1:115 | 0.85 | 21.3 | 1:23 |
| HSPC | 0.11 | 2.8 | 1:202 | 0.23 | 5.8 | 1:97 |
| DSPC | 0.10 | 2.5 | 1:184 | 0.58 | 14.5 | 1:32 |

### Example 5

A lipid formulation (DPPC:cholesterol in a ratio of 5:2 w/w) was dissolved in ethanol and added to a cisplatin solution. Part of the formulation was treated by cycles of cooling to 4 degrees Celsius and warming to 55 degrees celsius cycles while part was not treated thus. The lipid/cisplatin suspension was then washed by dialysis.

TABLE 5
Concentration of cisplatin with and
without cooling and warming cycles

| Starting | | | |
|---|---|---|---|
| concentration | | | Total |
| of Cisplatin | Concentration | Cooling & warming | concentration of |
| solution | of lipids | cycles | Cisplatin |
| 0.2 mg/ml | 1.4 mg/ml | No | Not Detectable |
| 0.2 mg/ml | 1.4 mg/ml | Yes | Not Detectable |
| 4.0 mg/ml | 28 mg/ml | No | 0.22 mg/ml |
| 4.0 mg/ml | 28 mg/ml | Yes | 0.46 mg/ml |

### Example 6

### Determination of Captured Volume of Cisplatin Vesicles of the Invention

The object was to determine the nature of the liposomal entrapped cisplatin (HLL cisplatin) by determining the concentration of the entrapped cisplatin within the liposome.

Vₜₒₜₐₗ=Vₗᵢₚₒₛₒₘₑ+V_{outside}

[Measurement of Vₗᵢₚₒₛₒₘₑ]

| | Abs at 450 nm | [dichromate] | V_{outside} | Vₗᵢₚₒₛₒₘₑ |
|---|---|---|---|---|
| HLL Cisplatin | 0.874 | 0.67 mg/ml | 1.88 ml | 0.12 ml |
| Saline only | 0.822 | 0.60 mg/ml | 2 ml | 0 ml |

Method: 1) 2 ml HLLCisplatin prepared as described in Example 4 was concentrated by centrifugation filter kit. 2) 0.8 ml of concentrated sample was recovered and 1.2 ml of 1 mg/ml dichromate was added to recover original volume. 0.8 ml normal saline+1.2 ml of dichromate was also prepared as a control. 3) Abs at 450 nm was measured to detect difference in dichromate concentration. To avoid turbidity from liposome sample, both samples were filtered by centrifugal filtration.

Result: 6% of total volume was occupied by liposomes.

Vₗᵢₚₒₛₒₘₑ=1.53 μL/μmoles lipid (total lipid 39.3 mM)

Next, Vₗᵢₚₒₛₒₘₑ=V_{captured}+V_{bilayer}

To estimate V_{bilayer}, the lamellarity of the vesicles of HLL cisplatin was determined.

[Measurement of lamellarity of HLL cisplatin vesicles]

| | | | % probe lipid at |
|---|---|---|---|
| | Fₜₒₜₐₗ | F_{inside} | outmost leaflet* |
| Fluorescence intensity | 14193 | 11349 | 20 |

*% probe lipid at outmost leaflet = ( Fₜₒₜₐₗ − F_{inside}) × 100 ÷ Fₜₒₜₐₗ

Method: Cisplatin vesicles were prepared with the method of Example 9 (1 liter batch) modified to add 0.5 wt % fluorescence probe lipid (NBD-PE). This probe lipid distributes evenly in membrane inside and outside. The ratio of amount of probes located in outmost membrane layer (surface of liposome) vs. the rest of probes is determined to estimate how many lipid layers exist in HLL Cisplatin. The ratio between probes located on liposome surface and probes located inside liposome was determined by adding a reducing agent dithionite to quench only surface probes. Then, total quenching was achieved by rupturing liposome with detergent.

Result: Outmost bilayer shell contains 40% of total lipids.

Based on geometric calculation, % lipid at outmost bilayer shell would be 52% and 36% for bi-lamellar and tri-lamellar vesicles, respectively. Therefore, it was concluded that the average lamellarity of HLL Cisplatin was three.

Assuming tri-lamellar vesicles, the ratio of Vₗᵢₚₒₛₒₘₑ/V_{captured} was calculated to be 1.2635. Therefore, the captured volume was:

$$\begin{matrix}{V}_{captured} = {V}_{liposome} \div 1.2635 \\ = 1.53 µL \text{/} µmoles lipid \div 1.2635 \\ = 1.21 µL \text{/} µmoles lipid \\ = 1.21 µL \text{/} µmoles lipid \times 39.3 mM ( total lipid concentration ) \\ = 47.6 µL \text{/} Ml\end{matrix}$$

The captured volume was 47.6 μL per every mL HLL Cisplatin and 4.76% of total volume. If entrapped cisplatin was assumed to be in an aqueous compartment of liposomes, its local cisplatin concentration would be estimated to be 21.0 mg/ml. This concentration was not only higher than cisplatin solubility limit at room temperature but more significantly it was much higher than initial charging concentration (4 mg/ml).

### Example 7

### Effect of Cooling Temperature on entrapment efficiency of HLL Cisplatin

The object was to find an optimum cooling temperature for the highest entrapment of cisplatin and avoid freezing and thawing. The resulting data helps optimize the manufacturing process.

| Post- | Actual | | | |
|---|---|---|---|---|
| infusion | temperature | | | |
| temperature | of the | Cooling and | [Cisplatin] | % Entrap- |
| treatment | sample | warming cycles | mg/ml | ment |
| Dry ice bath | frozen | 15 min cold & 15 | 0.34 | 8.5 |
| (−70° C.) | | min warm 6 cycles | | |
| Freezer | 0° C. | 15 min cold & 15 | 0.98 | 24.5 |
| (−20° C.) | | min warm 6 cycles | | |
| Ice bath | 4° C. | 15 min cold & 15 | 0.63 | 15.8 |
| (1° C.) | | min warm 6 cycles | | |

20 mg/ml DPPC, 8 mg/ml cholesterol, and 4 mg/ml cisplatin suspension was prepared by ethanol infusion. The sample was split to three equal aliquots which were treated by 6 cycles of cooling and warming using three different cooling temperatures. After a treatment of temperature cycles the samples were dialyzed to remove free cisplatin.

### Example 8

### Effect of Number of Temperature Cycles on Entrapment Efficiency

To determine an optimum number of temperature cycles for the most efficient entrapment of cisplatin. This will help determining the necessary process to achieve the most efficient entrapment of cisplatin.

| Number of | Low Lipids | | High Lipids | |
|---|---|---|---|---|
| Temper- | (7.5 mg/ml DPPC & | | (12.5 mg/ml DPPC & | |
| ature | 3 mg/ml cholesterol) | | 5 mg/ml cholesterol) | |
| cycles | [cisplatin] | % Entrapment | [cisplatin] | % Entrapment |
| 0 cycle | 0.05 mg/ml | 1.3 | 0.21 mg/ml | 5.3 |
| 1 cycle | 0.11 mg/ml | 2.8 | 0.23 mg/ml | 5.8 |
| 3 cycles | 0.39 mg/ml | 9.8 | 0.88 mg/ml | 22 |

Samples were prepared as in the previous example. At cooling the temperature of samples was 0° C. The temperature cycle was done by 15 min cooling and 15 min warming. The starting cisplatin concentration was 4 mg/ml and free cisplatin was removed by dialysis.

### Example 9

### Batch Scale and Process Efficiency

To determine if the efficiency of entrapment changed upon changing the size of the batch. The 20 mL batch was prepared as described in example 4. The IL batch was prepared indicated in the following steps:

1. Four grams of cisplatin were dissolved in 1 Liter of injection grade 0.9% sodium chloride at 65° C.

2. 20 grams of DPPC and 8 grams of cholesterol were dissolved in 120 mL of absolute ethanol at 65° C.

3. While mixing the cisplatin solution at 300 rpm (65° C.), the lipid solution was metered (infused) into the cisplatin solution at a flow rate of 20 mL/min.

4. After infusion, cisplatin/lipid dispersion was cooled down to −5° C. to 0° C. using a propylene glycol/water bath and kept for 45 minutes (cooling).

5. The dispersion was warmed up to 50° C. and maintained for 15 minutes (warming).

6. The cooling/warming cycle described in steps 4 and 5 was performed for two more times (three cycles total).

7. The dispersion was washed to remove free cisplatin by diafiltration. The permeate removing rate was 17-22 mL/min. The dispersion volume (1 L) was maintained constant by compensating the permeate with a feed of fresh sterile 0.9% sodium chloride solution.

The 200 mL batch was made in the same manner but employed 20% of the components.

The process efficiency was defined as the lipid/drug (wt/wt) ratio of initial ingredients divided by the lipid/drug ratio for the final product.

| | | | Lipid/drug | Lipid/drug | Process |
|---|---|---|---|---|---|
| Batch # | Batch size | | Pre-formation | Final product | efficiency |
| C3-18FT-04 | 20 | ml | 4.4 | 54.5 | 0.08 |
| C3-18FT-17 | 200 | ml | 5.85 | 27.3 | 0.21 |
| C3-18FT-19 | 200 | ml | 5.85 | 37.2 | 0.16 |
| C3-18FT-23 | 200 | ml | 5.85 | 36.9 | 0.16 |
| PC-1L-508 | 1 | L | 5.85 | 14.4 | 0.41 |
| CL-CISP-TN-01 | 1 | L | 7.0 | 19.2 | 0.36 |
| CL-CISP-TN-02 | 1 | L | 7.0 | 21.2 | 0.33 |

### Example 10

### Stability of Entrapped Lipid-Complexed Cisplatin

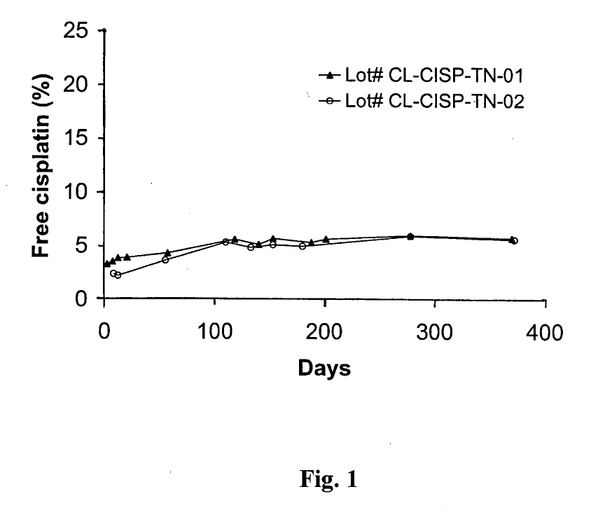

The stability of one liter batches of HLL cisplatin was monitored in time for the leakage of internal contents. The resulting data is presented in FIG. 1.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

1) A composition comprising a liposome or lipid complex and an entrapped active platinum compound, the liposome or lipid complex containing one or more lipids, wherein the active platinum compound to lipid ratio is from 1:50 to 1:2 by weight.

2) The composition of claim 1, wherein the active platinum compound to lipid ratio is from 1:50 to 1:5 by weight.

3) The composition of claim 1, wherein the active platinum compound to lipid ratio is from 1:50 to 1:10 by weight.

4) The composition of claim 1, wherein the active platinum compound is cisplatin.

5) The composition of claim 1, wherein the active platinum compound to lipid ratio is from 1:25 to 1:15 by weight.

6) The composition of claim 5, wherein the active platinum compound is cisplatin.

7) The composition of claim 6, the one or more lipids comprise DPPC.

8) The composition of claim 7, the one or more lipids comprise cholesterol.

9) The composition of claim 7, the one or more lipids comprise 50-100 [90?] mol % DPPC and 0-50 mol % cholesterol.

10) The composition of claim 7, the one or more lipids comprise 50-65 mol % DPPC and 35-50 mol % cholesterol.

11) A process for making a platinum aggregate comprising the steps of:
(a) combining an active platinum compound and a hydrophobic matrix carrying system;
(b) establishing the mixture at a first temperature; and
(c) thereafter establishing the mixture at a second temperature, which second temperature is cooler than the first temperature;
wherein the steps (b) and (c) are effective to increase the encapsulation of active platinum compound.

12) The process of claim 11, further comprising sequentially repeating the steps (b) and (c) for a total of two or more cycles.

13) The process of claim 11, wherein the active platinum compound solution is produced by dissolving active platinum compound in a saline solution to form a platinum solution.

14) The process of claim 13, wherein the active platinum compound is cisplatin

15) The process of claim 11, wherein the hydrophobic matrix carrying system comprises liposome or lipid complex-forming lipids.

16) The process of claim 15, wherein the one or more lipids comprise DPPC.

17) The process of claim 15, wherein the one or more lipids further comprise cholesterol.

18) The process of claim 11, wherein the hydrophobic matrix carrying system is produced by dissolving one or more lipids in ethanol to form a lipid solution and injecting the lipid solution into an aqueous medium containing active platinum compound.

19) The process of claim 11, further comprising sequentially repeating the steps (b) and (c) for a total of three or more cycles.

20) The process of claim 19, wherein the step (c) comprises establishing the mixture at a temperature from −25 degrees Celsius to 25 degrees Celsius.

21) The process of claim 19, wherein step (c) comprises establishing the mixture at a temperature from −5 degree Celsius to 5 degrees Celsius.

22) The process of claim 19, wherein the step (b) comprises establishing the mixture at a temperature from 4 degrees Celsius to 75 degrees Celsius.

23) The process of claim 19, wherein the step (b) comprises establishing the mixture at a temperature from 45 degrees Celsius to 55 degrees Celsius.

24) The process of claim 11, wherein the temperature differential between steps (b) and (c) is 25 degrees Celsius or more.

25) The process of claim 24, wherein the temperature established in step (b) is 50 degrees Celsius or more.

26) The process of claim 11, wherein the temperature established in step (b) is 50 degrees Celsius or more.

27) A platinum aggregate produced by the method of claim 11.

28) A platinum aggregate produced by the method of claim 14.

29) A pharmaceutical formulation comprising the composition of claim 1 and a pharmaceutically acceptable carrier or diluent.

30) A pharmaceutical formulation comprising the composition of claim 1, adapted for inhalation by a patient.

31) A pharmaceutical formulation comprising the composition of claim 1, adapted for injection into a patient.

32) The process of claim 11, further comprising, after all of steps (b) and steps (c) have been completed:
(d) removing un-entrapped active platinum compound by filtering through a membrane having a molecular weight cut-off selected to retain desired liposomes or lipid complexes and adding a liposome or lipid complex compatible liquid to wash out un-entrapped active platinum compound.
